(19) United States
(12) Patent Application Publication
(10) Pub. No.: US 2007/0010698 A1
(43) Pub. Date: Jan. 11, 2007

(54) Catalyst and process for the preparation of linear alkanes

(75) Inventor: Alberto De Angelis, Legnano (IT)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: POLIMERI EUROPA S.p.A, Brindisi (IT)

(21) Appl. No.: 10/545,801

(22) PCT Filed: Feb. 25, 2004
(86) PCT No.: PCT/EP04/02015

§ 371 Date: Jun. 27, 2006

(30) Foreign Application Priority Data
Feb. 27, 2003 (IT) MI2003A000347

(51) Int. Cl. C07C 5/03

(52) U.S. Cl. 585/277

(57) ABSTRACT

The present invention relates to a catalytic composition comprising at least one lanthanide, at least one metal belonging to group VIII B and a zeolite selected from zeolite Y and zeolite Y modified by the partial or total substitution of Si with Ti or Ge and/or the partial or total substitution of the aluminum with Fe, Ga or B. These catalytic compositions can be used in conversion processes of aromatics into linear alkanes.

The present invention relates to a catalytic composition comprising at least one lanthanide, at least one metal belonging to group VIII B and a zeolite selected from zeolite Y and zeolite Y modified by the partial or total substitution of Si with Ti or Ge and/or the partial or total substitution of the aluminum with Fe, Ga or B. These catalytic compositions can be used in conversion processes of aromatics into linear alkanes.

Aromatic compounds are one of the constituents of gasoline, whose concentration in the same is destined to decrease in the future. The legislation in force in Europe and in many other countries in the world is, in fact, tending to decrease the content of aromatic products in gasoline for environmental reasons, and consequently within a short period of time, there will be a considerable excess production of aromatic compounds, particularly those having 7 and 8 carbon atoms, which will not be easy to sell on the market.

A possible use of these aromatic compounds consists in their transformation, through hydrocracking catalyzed reactions, into alkanes, preferably linear, which represent an excellent feed for steamcrackers.

WO 01/27223 claims the use of zeolites, for this purpose, having a Spaciousness Index (S.I.) lower than 20, exchanged with hydrogenating metals. ZSM-5 exchanged with palladium proves to be the preferred zeolite.

With the use of this catalyst, a complete conversion of the model charge (toluene, cyclohexane or pseudo-cumene) is obtained, with a distribution of the reaction products ranging from methane to butanes. The content of methane—a by-product which cannot be subsequently processed by the steamcracker—in the mixture of reaction products, is about 5%. WO 01/27223 shows that large-pore zeolites, such as zeolite Y (S.I.=21), are not suitable for this reaction as they rapidly decay. When using zeolite Y in acid form, after only 8 hours of life, the conversion passes from 100% to 74%. On the contrary, the life of zeolite ZSM-5-Pd is reported to be of at least 10 hours. It has now been unexpectedly found that a catalytic composition containing zeolite Y, as such or modified, at least one lanthanide and a metal belonging to group VIII B, is an extremely active catalyst and, even more surprisingly, the life of this catalyst exceeds the best results obtained with the catalysts of the prior art, in particular those based on ZSM-5 and Palladium.

A first object of the present invention therefore relates to a catalytic composition comprising at least one lanthanide, at least one metal belonging to group VIII B and a zeolite selected from zeolite Y and zeolite Y modified by the partial or total substitution of Si with Ti or Ge and/or the partial or total substitution of the aluminum with Fe, Ga or B.

Zeolite Y was described for the first time in U.S. Pat. No. 3,130,007 and has the following formula, expressed as moles of oxides
0.9±0.2Na₂O•Al₂O₃•w SiO₂•x H₂O
wherein w has a value higher than 3 and up to 6 and x can be a value up to about 9. Its preparation is also described, for example, in “Verified Synthesis of Zeolitic materials” H. Robson Editor, Elsevier, second revised edition 2001, whereas the post-synthesis treatment to which the zeolite can be subjected, among which de-alumination, is described in “Introduction to Zeolite Science and Practice” chapter 5, H. van Bekkum et al. Editors, Studies in Surface Science and Catalysis, vol. 58, Elsevier. Zeolites Y with a molar ratio SiO₂/Al₂O₃ ranging from 3 to 400 can be used in the compositions of the present invention.

Modifications of zeolite Y, obtained by the partial or total isomorphic substitution of the aluminum of the zeolite with Fe, Ga or B, and/or the partial or total substitution of Si with Ti or Ge, can also be suitably used in the process of the present invention.

These modifications of zeolite Y can be prepared, for example, by substituting in the synthesis process of zeolite Y described in U.S. Pat. No. 3,130,007, a part of the sources of silicon and/or aluminum, with sources of Fe, Ga, B, Ti and/or Ge. Zeolite Y wherein Ge has fully substituted Si, is described in R. M. Barrer et al. J. Chem. Soc., 195-208 (1959) and in G. M. Johnson, Microporous and Mesoporous Material, 31, 195-204 (1999); zeolite Y wherein Si and Al have been completely substituted by Ge and Ga, are described in Barrer, J. Chem. Soc., 195-208 (1959).

The catalytic composition of the present invention, preferably contains the zeolite in its partially acid form, i.e. part of the cationic sites present in the zeolite are occupied by hydrogen ions.

The use of zeolite Y is a particularly preferred aspect. The molar ratio between silicon oxide and aluminum oxide in the crystalline lattice of zeolite Y based on silicon oxide and aluminum oxide preferably ranges from 5 to 50.

Lanthanum is the element belonging to the lanthanide group which is preferably used.

The lanthanide or lanthanides present in the catalytic composition can be in the form of an oxide or ion or a mixtures of these forms can be present. The quantity of lanthanide or lanthanides, expressed as an element, can vary from 0.5 to 20% by weight, preferably between 1 and 15% by weight, with respect to the total weight of the catalytic composition.

The metal of group VIII B is preferably selected from platinum and palladium, and is preferably palladium. The metal of group VIII B can be present in the catalytic composition in the form of an oxide, ion, metal or a mixture of these forms. The quantity of metal of group VIII B, expressed as an element, can vary from 0.001 and 10% by weight, preferably from 0.1 to 5% by weight, with respect to the total weight of the catalytic composition.

The catalytic composition of the present invention is preferably prepared by introducing into the zeolite, first the lanthanide and then the metal of group VIII B.

The metal of group VIII B and the lanthanide can be introduced into the catalytic composition by treating the zeolite, preferably in acid form, with a lanthanide compound and a compound of the metal of group VIII B. When the catalytic composition of the present invention contains more than one lanthanide, or more than one metal of group VIII B, a mixture of compounds of these elements will be used in its preparation.

Any of the known techniques can be used for introducing the lanthanide, such as exchange in the solid state with a lanthanide salt, ion exchange in an aqueous solution, or impregnation. Ion exchange or impregnation is preferably used. In the former case, the zeolite, preferably in acid form, is treated with an aqueous solution of a lanthanum salt having a concentration which can vary from 0.1 to 10 M, preferably from 0.1 to 1.0 M, for example an 0.1-0.5 M aqueous solution of the corresponding nitrate, citrate, acetate, chloride or sulfate, under reflux for 1-24 hours. After suitable washings with distilled water, the sample resulting from the ion exchange is dried and then calcined at a temperature ranging from 400 to 600° C. for 1-10 hours. When the lanthanide is introduced by impregnation, the known technique of wet imbibition is used, followed by drying and calcination as in the case of ion exchange.

An at least partial transformation of the lanthanide ion into the corresponding oxide will take place as a result of the calcination.

Ion exchange is the technique preferably used for introducing the lanthanide.

The metal of group VIII B can be introduced by means of ion exchange or impregnation, into the zeolite containing the lanthanide, prepared in the previous step using one of the above techniques.

In the former case, the composition containing the zeolite and the lanthanide is treated with an aqueous solution of a salt of the metal of group VIII B, for example an aqueous solution having a concentration of 0.01-5 M of a corresponding complex, preferably a concentration of 0.01-0.5 M. The sample resulting from the ion exchange is dried, after suitable washings, and then calcined at a temperature ranging from 400 and 600° C. for 1-10 hours.

When the metal of group VIII B is introduced by impregnation, the known wet imbibition technique is used, followed by drying and calcination, as in the case of ion exchange.

An at least partial transformation of the metal ion of group VIII B into the corresponding oxide will take place as a result of the calcination.

Impregnation is the technique preferably used for introducing the metal of group VIII B.

Calcination between the introduction step of the first element and the introduction step of the second element is optional; if calcination is not effected, the partial transformation of the metal ions into the corresponding oxides will take place contemporaneously during the calcination carried out at the end of the second step.

According to a particularly preferred aspect, the catalytic compositions of the present invention are prepared by depositing the lanthanide on the zeolite in acid form, through ion exchange, optionally calcining the product thus obtained, subsequently depositing the metal of group VIII B by ion exchange and calcining the product obtained.

The compositions thus prepared, including a zeolite Y exchanged with at least one lanthanide, and at least a metal of group VIII B, prove to have the best results in terms of activity and duration. Catalytic compositions containing zeolite Y exchanged with lanthanum and palladium are particularly preferred.

After the synthesis step, an at least partial reduction of the ion of the metal of group VIII B to the corresponding elements, can be effected. The reduction to the metal can be obtained by treating the catalytic composition with hydrogen or with a reducing agent, and it can be effected on the catalytic composition before its use or in the same reactor in which the catalytic composition will be used.

The catalytic composition of the present invention can be used in a mixture with suitable binders, such as silica, alumina, clay. The catalytic composition and the binder are mixed in proportions ranging from 50:50 to 95:5, preferably 60:40 and 90:10. The mixture of the two components is prepared in the desired end-form, for example cylindrical extruded rods or other known forms.

The above catalytic compositions can be used in processes for the conversion of aromatic compounds into alkanes.

A further object of the present invention therefore relates to a process for the conversion of aromatic compounds into linear alkanes, which comprises putting a mixture containing aromatic compounds in contact with a catalytic composition including at least one lanthanide, at least one metal belonging to group VIII B and one zeolite selected from zeolite Y and zeolite Y modified by the partial or total substitution of Si with Ti or Ge and/or the partial or total substitution of the aluminum with Fe, Ga or B.

Fractions coming from thermal or catalytic conversion plants, fractions of mineral oil rich in aromatic compounds, such as, for example, gasoline from pyrolysis (Pygas), fractions coming from pyrolysis gasoline, fractions coming from plants for the production of aromatic compounds, are mixtures containing aromatic compounds which are suitable for being treated according to the process of the present invention.

These charges can be optionally mixed with heavier fractions, coming, for example, from fuel oil from steam cracking (FOK) or Light Cycle Oil (LCO) from fluid bed catalytic cracking. As these heavy fractions contain sulfur, which is known to be a poison for hydrogenation catalysts, an unpredictable and extremely favourable aspect consists in the fact that the catalytic compositions of the present invention do not undergo any deactivation due to the presence of sulfur and are therefore suitable for processing mixtures of aromatic hydrocarbons also containing heavy fractions, such as FOK and LCO.

Pyrolysis gasoline is a by-product of the steam cracking process, wherein ethylene and propylene are obtained from light hydrocarbon cuts, such as straight-run naphtha (petroleum fraction substantially containing C₅ and C₆ hydrocarbons), LPG (“Liquefied Petroleum gas”, a petroleum fraction containing C₃ and C₄ hydrocarbons), propane or ethane.

The mixtures containing aromatic compounds which can be subjected to the process of the present invention, and in particular pyrolysis gasoline, prevalently contain toluene, ethyl benzene, xylenes, benzene, C₉ aromatic compounds, naphthalene derivatives and their mixtures. The naphthalene derivatives can, for example, be naphthalene, methyl naphthalene, dimethyl naphthalene, trimethyl naphthalene and/or tetramethyl naphthalene.

The mixtures which are treated with the process of the present invention can additionally contain cyclic alkanes and linear and/or cyclic alkenes.

According to a preferred aspect of the present invention, the resulting fraction of n-alkanes resulting from the process of the present invention, with the exclusion, therefore, of methane and hydrogen, ranges from 50 to 90%.

According to an aspect of the present invention, the resulting fraction of n-alkanes is mainly composed of ethane, propane, n-butane and n-pentane.

A preferred aspect of the present invention is to use a catalytic composition wherein the zeolite is in partially acid form, i.e. a portion of the cationic sites are occupied by hydrogen ions. Zeolite Y is preferred among the zeolites which can be used.

Catalytic compositions in which the element belonging to the lanthanide group is lanthanum and the metal of group VIII B is selected from platinum and palladium, are preferred. Catalytic compositions containing zeolite Y exchanged with lanthanum and palladium are particularly preferred.

The process of the present invention is carried out in the presence of hydrogen at a pressure of 5 to 200 bar, preferably between 50 and 70 bar, at a temperature ranging from 150° C. to 550° C., preferably from 300° C. to 500° C. The process is preferably carried out in continuous in a fixed or fluid bed reactor, in gas or partially liquid phase, at a WHSV (Weight Hourly Space Velocity, expressed as kg of charge/hour/kg of catalyst) of between 0.1 and 20 hours⁻¹, preferably between 0.5 and 3 hours⁻¹.

The composition of the present invention is activated, before use, in nitrogen at a temperature ranging from 300 to 700° C., for a time ranging from 1 to 24 hours and a pressure of between 0 and 10 barg.

An activation with hydrogen at a temperature of 300-700° C., a pressure of 0-10 barg, for a time of 1 to 24 hours, can be effected in addition to or as a substitution of the preceding one.

Some illustrative examples are provided for a better understanding of the present invention, which however should in no way be considered as limiting the scope of the invention itself.

### EXAMPLE 1

Synthesis of Zeolite Y with Lanthanum (Y-La)

25 g of commercial zeolite Y (Toyosoda HSZ 320 HOA) with a molar ratio SiO₂/Al₂O₃ equal to 5.5 and a sodium content as oxide (Na₂O) of 4%, and 500 ml of a 2 molar aqueous solution of ammonium nitrate are charged into a glass flask. The suspension is left at reflux temperature for three hours, under stirring. After this period the mixture is filtered on a Buckner vacuum funnel, dried in an oven and calcined at a temperature of 550° C. in air for 5 hours, obtaining a zeolite Y in acid form. The solid thus obtained is charged into the glass flask and 500 ml of an 0.2 molar solution of lanthanum nitrate hexahydrate are added. The solution is left at reflux temperature for three hours, under stirring. At the end of this period, the suspension is filtered on a Buckner vacuum funnel, the filtrate is washed with distilled water and dried in an oven. The above operation is repeated three times, for a total of four exchanges with a solution of lanthanum nitrate.

After the last exchange, the solid is dried in an oven, and is then calcined in a muffle at 550° C. under an air flow. A zeolite Y is obtained, exchanged with lanthanum, with a molar ratio SiO₂/Al₂O₃ equal to 5.6, a molar ratio La₂O₃/Al₂O₃ equal to 0.22 and a molar ratio Na₂O/Al₂O₃ equal to 0.0096.

### EXAMPLE 2

Synthesis of Zeolite Y with Lanthanum and Palladium (Y-La-Pd 2.5%)

20 g of zeolite Y containing lanthanum and prepared according to example 1 are charged into a beaker, and 160 ml of distilled water and 12.70 g of a solution at 4.41% by weight of [Pd(NH₃)₄] (NO₃)₂ are added. The zeolite suspension is stirred for 4 hours at room temperature, filtered on a Buckner vacuum funnel and the filtrated solid is dried in an oven at 150° C. overnight. The product is then calcined in a muffle at 400° C. for 12 hours in air.

A zeolite Y is obtained containing lanthanum in an amount equal to 4.03% by weight, and palladium at 2.7% by weight.

### EXAMPLE 3

Synthesis of Zeolite Y with Lanthanum and Palladium (Y-La-Pd 0.3%)

20 g of zeolite prepared according to example 1 are charged into a beaker, and 160 ml of distilled water and 1.3 g of a solution at 4.41% by weight of [Pd(NH₃)₄] (NO₃)₂ are added. The zeolite suspension is stirred for 4 hours at room temperature, filtered on a Buckner vacuum funnel and the filtrated solid is dried in an oven at 150° C. overnight. The product is then calcined in a muffle at 400° C. for 12 hours in air.

A zeolite Y is obtained containing lanthanum in an amount of 6.26% by weight, and palladium at 0.35% by weight.

### EXAMPLE 4

Catalytic Test

3 g of catalyst prepared according to example 2 are charged into a steel reactor, which is heated to 400° C., the catalyst is activated by feeding hydrogen, and the reaction mixture is then fed in gas phase, at a pressure of 60 barg, consisting of pseudo-cumene (1,2,4 trimethyl benzene) and hydrogen: the molar ratio of the feed is 1 (pseudo-cumene) to 78 (hydrogen). The feed is effected at a WHSV of 0.7 hours⁻¹, referring to pseudo-cumene alone.

The gases leaving the reactor are sampled at different reaction times and analyzed by means of gas chromatography. The pseudo-cumene conversion is always equal to 100%.

Liquid compounds are never found among the reaction products.

The weight percentage composition of the mixture of reaction products (with the exclusion of the non-converted hydrogen) is indicated in table 1. In this reaction mixture, n-butane forms 54.36% of all the butanes and n-pentane forms 35.76% of all the pentanes.

TABLE 1

| | | | | | | Hexanes |
|---|---|---|---|---|---|---|
| | | | | | | and heavy |
| Reaction | Methane | Ethane | Propane | Butanes | Pentanes | products |
| time | % weight in | % weight in | % weight in | % weight in | % weight in | % weight in |
| (hours) | react. mixt. | react. mixt. | react. mixt. | react. mixt. | react. mixt. | react. mixt. |
| 7 | 2.33 | 6.99 | 29.45 | 44.89 | 16.34 | 0 |
| 24 | 2.08 | 6.12 | 28.69 | 44.72 | 18.37 | 0 |
| 31 | 2.09 | 6.49 | 29.58 | 44.60 | 17.23 | 0 |
| 48 | 2.12 | 5.98 | 29.21 | 45.03 | 17.68 | 0 |

### EXAMPLE 5

Catalytic Test

3 g of catalyst prepared according to example 2 are charged into a steel reactor, which is heated to 430° C., the catalyst is activated by feeding hydrogen, and the reaction mixture is then fed in gas phase, at a pressure of 60 barg, consisting of pseudo-cumene (1,2,4 trimethyl benzene) and hydrogen: the molar ratio of the feed is 1 (pseudo-cumene) to 78 (hydrogen). The feed is effected at a WHSV of 0.7 hours⁻¹, referring to pseudo-cumene alone.

The gases leaving the reactor are sampled at different reaction times and analyzed by means of gas chromatography. The pseudo-cumene conversion is always equal to 100%.

Liquid compounds are never found among the reaction products.

The weight percentage composition of the reaction products (with the exclusion of the non-converted hydrogen) is shown in table 2.

TABLE 2

| | | | | | | Hexanes |
|---|---|---|---|---|---|---|
| | | | | | | and heavy |
| Reaction | Methane | Ethane | Propane | Butanes | Pentanes | products |
| time | % weight in | % weight in | % weight in | % weight in | % weight in | % weight in |
| (hours) | react. mixt. | react. mixt. | react. mixt. | react. mixt. | react. mixt. | react. mixt. |
| 7 | 12.22 | 10.63 | 34.49 | 36.76 | 5.89 | 0 |
| 24 | 8.88 | 9.81 | 34.58 | 39.48 | 7.24 | 0 |
| 31 | 8.29 | 9.83 | 34.49 | 39.74 | 7.65 | 0 |
| 48 | 6.99 | 9.23 | 33.68 | 41.14 | 8.95 | 0 |

### EXAMPLE 6

Catalytic Test

3 g of catalyst prepared according to example 3 are charged into a steel reactor, which is heated to 400° C., the catalyst is activated by feeding hydrogen, the reaction mixture is then fed in gas phase, at a pressure of 60 barg, consisting of pseudo-cumene (1,2,4 trimethyl benzene) and hydrogen: the molar ratio of the feed is 1 (pseudo-cumene) to 78 (hydrogen). The feed is effected at a WHSV of 0.7 hours⁻¹, referring to pseudo-cumene alone.

The gases leaving the reactor are sampled at different reaction times and analyzed by means of gas chromatography. The pseudo-cumene conversion is always equal to 100%.

Liquid compounds are never found among the reaction products.

The weight percentage composition of the reaction products (with the exclusion of the non-converted hydrogen) is shown in table 3.

TABLE 3

| | | | | | | Hexanes |
|---|---|---|---|---|---|---|
| | | | | | | and heavy |
| Reaction | Methane | Ethane | Propane | Butanes | Pentanes | products |
| time | % weight in | % weight in | % weight in | % weight in | % weight in | % weight in |
| (hours) | react. mixt. | react. mixt. | react. mixt. | react. mixt. | react. mixt. | react. mixt. |
| 7 | 2.25 | 9.45 | 29.21 | 44.32 | 14.96 | 0 |
| 24 | 2.24 | 7.81 | 28.78 | 44.12 | 16.01 | 0 |
| 31 | 2.12 | 8.28 | 28.51 | 44.18 | 15.98 | 0 |
| 48 | 2.02 | 7.74 | 28.89 | 43.63 | 16.46 | 1.26 |

No product was found with a molecular weight higher than hexane.

### EXAMPLE 7

Catalytic Test

3 g of catalyst prepared according to example 3 are charged into a steel reactor, which is heated to 430° C., the catalyst is activated by feeding hydrogen, the reaction mixture is then fed in gas phase, at a pressure of 60 barg, consisting of pseudo-cumene (1,2,4 trimethyl benzene) and hydrogen: the molar ratio of the feed is 1 (pseudo-cumene) to 78 (hydrogen). The feed is effected at a WHSV of 0.7 hours⁻¹, referred to pseudo-cumene alone.

The gas leaving the reactor is sampled at different reaction times and analyzed by means of gas chromatography. The pseudo-cumene conversion is always equal to 100%.

Liquid compounds are never found among the reaction products.

The weight percentage composition of the reaction products (with the exclusion of the non-converted hydrogen) is shown in table 4.

TABLE 4

| | | | | | | Hexanes |
|---|---|---|---|---|---|---|
| | | | | | | and heavy |
| Reaction | Methane | Ethane | Propane | Butanes | Pentanes | products |
| time | % weight in | % weight in | % weight in | % weight in | % weight in | % weight in |
| (hours) | react. mixt. | react. mixt. | react. mixt. | react. mixt. | react. mixt. | react. mixt. |
| 7 | 6.27 | 12.98 | 35.00 | 37.31 | 8.43 | 0 |
| 24 | 4.70 | 11.05 | 34.22 | 38.77 | 11.30 | 0 |
| 31 | 4.15 | 11.41 | 34.27 | 38.86 | 11.32 | 0 |
| 48 | 3.68 | 10.95 | 34.00 | 39.06 | 12.31 | 0 |

### EXAMPLE 8

Catalytic Test

3 g of catalyst prepared according to example 2 are charged into a steel reactor, which is heated to 430° C., the catalyst is activated by feeding hydrogen, the reaction mixture is then fed in gas phase, at a pressure of 60 barg, consisting of an organic phase and hydrogen, in the ratios specified in the previous examples. Said organic phase consists of pseudo-cumene (1,2,4 trimethyl benzene) for 80% by weight and 2-methyl naphthalene for the remaining 20%. Commercial 2-methyl naphthalene is contaminated by a sulfur content of 10,000 ppm and consequently the sulfur content in the organic phase is equal to 2,000 ppm. The feed is effected at a WHSV of 0.7 hours⁻¹, referring to the organic phase.

The gases leaving the reactor are sampled at different reaction times and analyzed by means of gas chromatography. The conversion of the organic phase is always equal to 100%.

Liquid compounds are never found among the reaction products.

The weight percentage composition of the reaction products (with the exclusion of the non-converted hydrogen) is shown in table 5.

TABLE 5

| | | | | | | Hexanes |
|---|---|---|---|---|---|---|
| | | | | | | and heavy |
| Reaction | Methane | Ethane | Propane | Butanes | Pentanes | products |
| time | % weight in | % weight in | % weight in | % weight in | % weight in | % weight in |
| (hours) | react. mixt. | react. mixt. | react. mixt. | react. mixt. | react. mixt. | react. mixt. |
| 7 | 3.92 | 6.04 | 32.29 | 42.85 | 14.90 | 0 |
| 24 | 2.46 | 5.00 | 31.43 | 44.83 | 16.27 | 0 |
| 31 | 2.41 | 4.92 | 31.19 | 44.51 | 16.97 | 0 |
| 48 | 2.25 | 4.92 | 30.43 | 44.97 | 17.44 | 0 |
| 55 | 2.15 | 4.82 | 30.03 | 45.02 | 17.98 | 0 |
| 72 | 2.09 | 4.85 | 30.67 | 45.49 | 16.88 | 0 |

### EXAMPLE 9

Life Test with Zeolite Y-La-Pd (2.5%)

3 g of catalyst prepared according to example 2 are charged into a steel reactor, which is heated to 430° C., the catalyst is activated by feeding hydrogen, the reaction mixture is then fed in gas phase, at a pressure of 60 barg, consisting of pseudo-cumene (1,2,4 trimethyl benzene) and hydrogen: the molar ratio of the feed is 1 (pseudo-cumene) to 78 (hydrogen). The feed is effected at a WHSV of 0.7 hours⁻¹, referring to pseudo-cumene alone.

The gases leaving the reactor are sampled at different reaction times and analyzed by means of gas chromatography. The pseudo-cumene conversion is shown in table 6 below. The table also indicates, for comparison, the conversion data of zeolite YH⁺ described in WO 01/27223, page 13, Table 2.

TABLE 6

| Reaction time (hours) | Y—La—Pd | YH⁺ |
|---|---|---|
| 0.5 | 100 | 100 |
| 8 | 100 | 74 |
| 24 | 100 | — |
| 31 | 100 | — |
| 48 | 100 | — |
| 55 | 100 | — |
| 72 | 100 | — |
| 79 | 100 | — |
| 96 | 100 | — |

1. A catalytic composition comprising at least one lanthanide, at least one metal belonging to group VIII B and a zeolite selected from zeolite Y and zeolite Y modified by the partial or total substitution of Si with Ti or Ge and/or the partial or total substitution of aluminum with Fe, Ga or B.

2. The composition according to claim 1, wherein the zeolite is zeolite Y.

3. The catalytic composition according to claim 1, wherein the zeolite is partially in acid form.

4. The catalytic composition according to claim 2, wherein the molar ratio between silicon oxide and aluminum oxide ranges from 3 to 400.

5. The catalytic composition according to claim 4, wherein the molar ratio between silicon oxide and aluminum oxide ranges from 5 to 50.

6. The catalytic composition according to claim 1, wherein the lanthanide is present in the form of an oxide or ion or a mixture of these two forms.

7. The catalytic composition according to claim 1, wherein the quantity of lanthanide, expressed as an element, varies from 0.5 to 20% by weight.

8. The catalytic composition according to claim 7, wherein the quantity of lanthanide, expressed as an element, varies from 1 to 15% by weight.

9. The catalytic composition according to claim 1, wherein the lanthanide is lanthanum.

10. The catalytic composition according to claim 1, wherein the metal of group VIII B is present in the form of an oxide, ion, metal or a mixture of these forms.

11. The catalytic composition according to claim 1, wherein the quantity of metal of group VIII B, expressed as an element, ranges from 0.001 to 10% by weight.

12. The catalytic composition according to claim 11, wherein the quantity of metal of group VIII B, expressed as an element, ranges from 0.1 to 5% by weight.

13. The catalytic composition according to claim 1, wherein the metal of group VIII B is selected from platinum and palladium.

14. The catalytic composition according to claim 1, containing a binder selected from silica, alumina and clay, in a weight proportion ranging from 50:50 to 95:5.

15. The catalytic composition according to one or more of the previous claims, wherein the zeolite is zeolite Y, the lanthanide is lanthanum and the metal of group VIII B is selected from platinum and palladium.

16. The catalytic composition according to claim 1, containing the zeolite exchanged with at least one lanthanide and at least one metal of group VIII B.

17. The catalytic composition according to claim 15 and 16, comprising a zeolite Y exchanged with lanthanum and palladium.

18. A process for the preparation of the catalytic composition according to claim 1, which comprises treating the zeolite with a lanthanide compound, treating the product thus obtained with a metal of group VIII B, drying and calcining.

19. The process according to claim 18, wherein the zeolite is in acid form.

20. The process according to claim 18, wherein the treatment with a lanthanum compound and the treatment with a compound of a metal of group VIII B are selected from ion exchange and impregnation.

21. The process according to claim 20, wherein the ion exchange and impregnation are effected using an aqueous solution of a lanthanide salt and an aqueous solution of a salt of a metal of group VIII B.

22. The process according to claim 21, wherein the lanthanide salt is selected from the corresponding nitrates, citrates, acetates, sulfates or chlorides.

23. The process according to one or more of the claims from 18 to 22, which comprises the treatment of the zeolite in acid form through ion exchange with an aqueous solution of a lanthanide salt, drying, optionally calcining the resulting product, treating it with an aqueous solution of a salt of a metal of group VIII B by means of ion exchange, drying and calcining.

24. A process for the preparation of linear alkanes which comprises putting a mixture containing aromatic compounds in contact with a catalytic composition, according to one or more of the claims from 1 to 17.

25. The process according to claim 24, wherein the mixture containing aromatic compounds is selected from fractions coming from thermal or catalytic conversion plants and fractions of mineral oils.

26. The process according to claim 25, wherein said fractions are gasoline from pyrolysis, fractions coming from pyrolysis gasoline, fractions coming from aromatic production plants.

27. The process according to claims 24, 25 or 26, wherein the aromatic products are toluene, ethyl benzene, xylenes, benzene, C₉ aromatic compounds, naphthalene derivatives, and mixtures thereof.

28. The process according to claims 24, 25 or 26, wherein the mixtures containing the aromatic compounds also contain cyclic alkanes and linear and/or cyclic alkenes.

29. The process according to claims 24, 25 or 26, wherein said charges are mixed with heavier fractions, coming from fuel oil from steam cracking (FOK) or Light Cycle Oil (LCO) from fluid bed catalytic cracking.

30. The process according to claim 24, wherein the resulting fraction of linear alkanes mainly consists of ethane, propane, n-butane and n-pentane.

31. The process according to claim 24, carried out in the presence of hydrogen at a pressure ranging from 5 to 200 bar, preferably between 50 and 70 bar, at a temperature ranging from 150 to 550° C.

32. The process according to claim 31, carried out at a pressure ranging from 50 to 70 bar, at a temperature ranging from 300 to 500° C.

33. The process according to claim 24, carried out in continuous, in a fixed or fluid bed reactor, in gas or partially liquid phase, at a WHSV of between 0.1 and 20 hours^{31 1}.

34. The process according to claim 33, carried out at a WHSV of between 0.5 and 3 hours⁻¹.

35. The process according to claim 24, wherein the catalytic composition is activated, before use, under nitrogen, at a temperature ranging from 300 to 700° C., for a time ranging from 1 to 24 hours, at a pressure ranging from 0 to 10 barg.

36. The process according to claim 24, wherein the catalytic composition is activated, before use, with hydrogen, at a temperature ranging from 300 to 700° C., at a pressure ranging from 0 to 10 barg, for a time ranging from 1 to 24 hours.

37. Zeolite Y modified by the partial substitution of Si with Ti or Ge and/or the partial substitution of aluminum with Fe, Ga or B.
